(19) United States
(12) Patent Application Publication
(10) Pub. No.: US 2011/0054333 A1
(43) Pub. Date: Mar. 3, 2011

(54) Stent Flow Sensor

(75) Inventor: Lee Hoffer, Boynton Beach, FL (US)

(73) Assignee: STENTRONICS, INC., Boynton Beach, FL (US)

(21) Appl. No.: 12/550,149

(22) Filed: Aug. 28, 2009

(51) Int. Cl. A61B 5/0265; A61F 2/06

(52) U.S. Cl. 600/505; 623/1.16

(57) ABSTRACT

A stent for placement in a blood vessel of a patient, the stent including: a proximal end, a distal end, and a generally circular cross-section with a diameter; a Micro Electro-Mechanical System (MEMS) ultrasound sensor using a Doppler principle for determining patency and flow rate through the cross-section, and pressure drop from the proximal end to the distal end; a transmitter for providing signals to an external receiver outside the patient's body; and a coil device for receiving energy from outside the patient's body and coupled to the transmitter for powering the transmitter.

### CROSS-REFERENCE TO RELATED APPLICATIONS

None.

### STATEMENT REGARDING FEDERALLY SPONSORED-RESEARCH OR DEVELOPMENT

None.

### INCORPORATION BY REFERENCE OF MATERIAL SUBMITTED ON A COMPACT DISC

None.

### FIELD OF THE INVENTION

The invention disclosed broadly relates to the field of arterial stents and more particularly relates to the field of integrating a blood flow sensor with a stent.

### BACKGROUND OF THE INVENTION

The idea of an integrated sensor placed into a stent has been addressed in the past. However, up until now no actually functional sensor has been reported. Transfer of energy from the outside to an extracorporeal system has been addressed by groups performing research and development towards eye prostheses, i.e., electronic devices capable of simulating the optical nerves. The energy supply requires miniaturized systems as they have to be placed inside the skull. However, many principles of energy transfer are also widely addressed by artificial heart research groups. More recently MEMs (micro electromechanical systems) technology dealing with sensor to sensor communication and energy harvesting for actuator power supply focus on that issue.

### SUMMARY OF THE INVENTION

Briefly, according to an embodiment of the invention a stent for placement in a blood vessel of a patient includes a proximal end, a distal end, and a generally circular cross-section of the stent; a Micro Electro-Mechanical System (MEMS) ultrasound sensor using the Doppler principle for determining patency and flow rate through the cross-section, and pressure drop from the proximal end to the distal end; a transmitter for providing signals to an external receiver outside the patient's body; and a coil device for receiving energy from outside the patient's body and coupled to the transmitter for powering the transmitter.

### BRIEF DESCRIPTION OF THE DRAWINGS

To describe the foregoing and other exemplary purposes, aspects, and advantages, we use the following detailed description of an exemplary embodiment of the invention with reference to the drawings, in which:

FIG. 1a is an illustration of a stent design according to an embodiment of the present invention;

FIG. 1b is an illustration of a stent design with a flow sensor attached;

FIG. 2 is a simplified block diagram of a stent sensor, according to an embodiment of the present invention;

FIG. 3 is an illustration of a stent vessel arrangement, according to an embodiment of the present invention;

FIG. 4 is a top level block diagram of the stent sensor power supply, according to an embodiment of the present invention;

FIG. 5 is an illustration of a receive coil wound circumferentially around the stent, according to an embodiment of the present invention;

FIG. 6 shows a power recovery and regulator module, according to an embodiment of the present invention;

FIG. 7 shows a block diagram of a CW-Doppler subsystem configured to operate according to an embodiment of the present invention;

FIG. 8 shows a cross-sectional and axial view of a transducer stent mounting, according to an embodiment of the present invention;

FIG. 9 shows a stent transducer arrangement for a transit time flow meter, according to an embodiment of the present invention;

FIG. 10 shows a 1-3 Piezo composite transducer material and electrode arrangement according to an embodiment of the present invention;

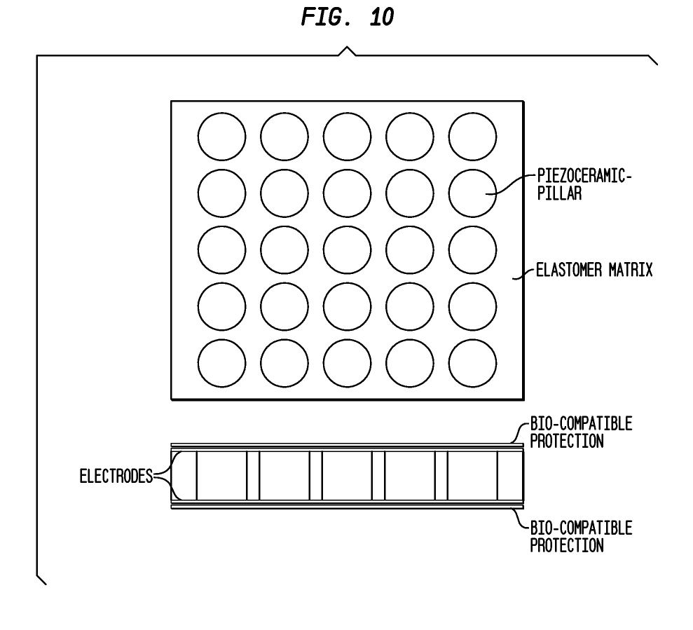

FIG. 11 shows a transmit/receive transducer equivalent circuit, according to an embodiment of the present invention;

FIG. 12 shows a basic bending experiment with PiezoLab Morph, according to an embodiment of the present invention; and

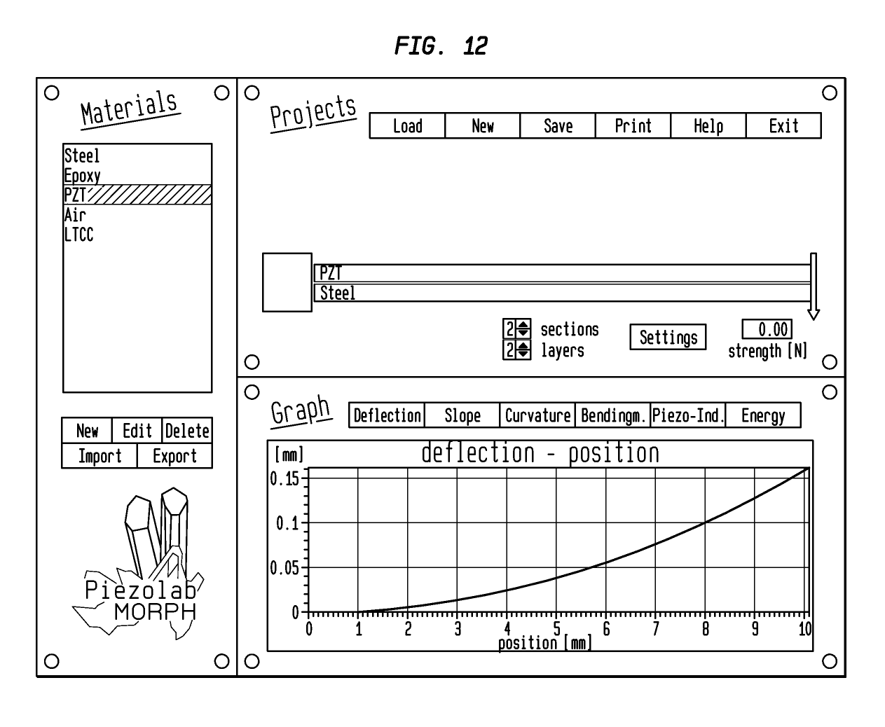

FIG. 13 shows bender displacement, according to an embodiment of the present invention.

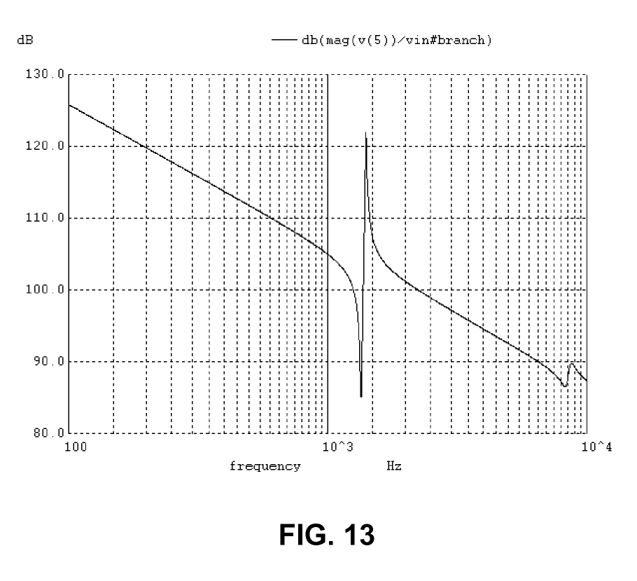

While the invention as claimed can be modified into alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the scope of the present invention.

### DETAILED DESCRIPTION

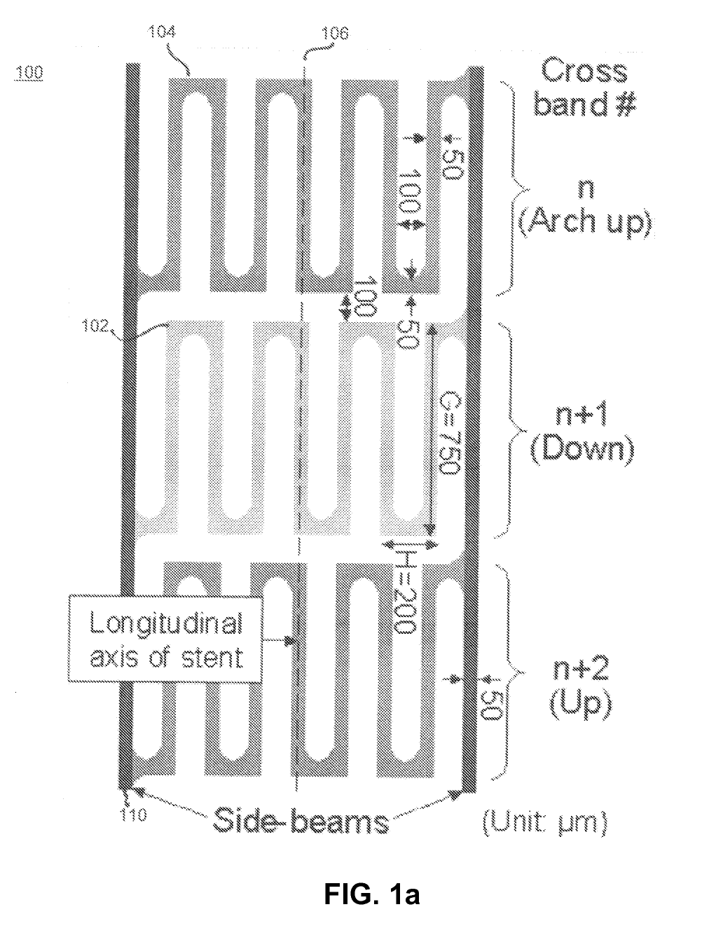

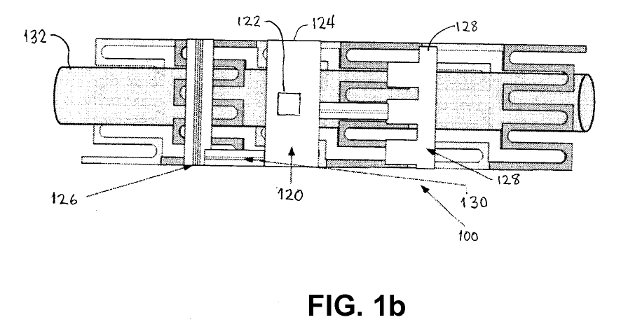

FIG. 1a illustrates a stent design 100 according to an embodiment of the present invention. The stent 100 comprises a plurality of stent segments 102 and 104 aligned along a longitudinal axis 106 from a proximal end to a distal end of the stent 100. The stent segments 102 and 104 are interlocked along the circumference of the stent 100, forming a mesh. Side beams 110 are found along the outside wall of the stent 100. The stent has a generally circular cross-section.

FIG. 1b shows the stent 100 with a stent flow sensor 120 attached thereto. Electronic analog/digital circuitry 122 is mounted in a flex tape 124 and bonded onto the stent crossbar. The flow sensor also comprises a transducer element which attaches to the stent 100 and slides over the stent segments. The transducer element comprises a receive coil 126 wound around in the flex tape 124 fixed over the stent crossbar. A flex tape interconnect 130 connects different modules to each other. An alternative to the coil is a mechanical bending vibrator. The receive coil 126 senses the flow of blood thorough the stent which induces a current in the coil that is processed by the circuitry. The components of the transducer are interconnected by flex tape interconnect modules 128. A balloon catheter 132 is located inside the stent to expand the stent when it is over the desired area.

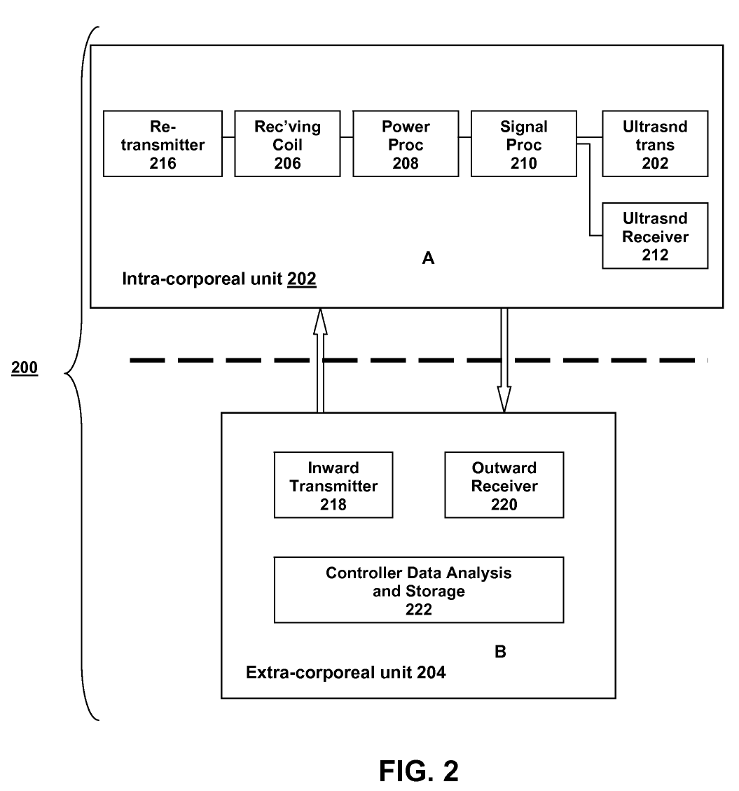

In FIG. 2 a simplified block diagram illustrates a blood flow sensor 200 integrated into the stent 100 for long term monitoring of stent function. The blood flow sensor 200 includes two main components: the intra-corporeal unit 202 placed in the stent 100 and the extra-corporeal unit 204 located outside the body of the patient. The intra corporeal unit 202 consists of the following components: a pick up coil 206, a power processing unit 208, a signal processing unit 210, the ultrasound flow sensor units (212 and 214), and the re-transmitting unit (216).

The extra-corporeal unit 204 consists of an inward transmitting coil 218 and the outward receiving coil 220. In addition, a controller 222 is used for timing data flow as well as analyzing transmitted data, estimating flow parameters and storing results.

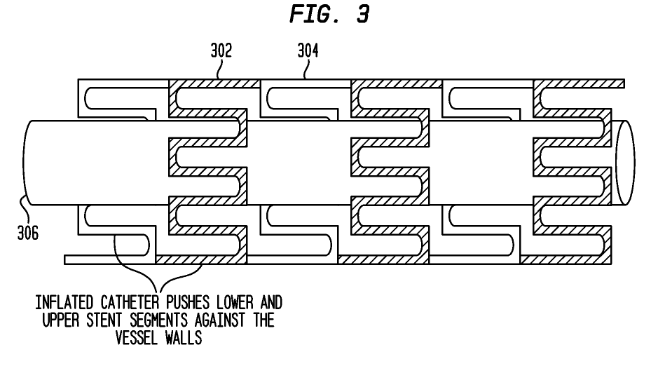

The intra-corporeal components are built into individual boxes, of approximately 1 mm in length, 0.5 mm in height and approximately 2 mm wide, shaped into a circular form matching the internal vessel wall as depicted with reference to FIG. 3.

FIG. 3 depicts a mesh stent showing how the stent is formed from an alternating sequence of approximately 1 mm long segments forming the upper (dark shading) 302 and lower (light shading) 304 segments of the stent 100. Upper and lower in this example refers to the positioning of the stent segments around the catheter 306 in this plan view. As is shown here, an inflated catheter is positioned between the alternating segments, pushing the lower and upper stent segments against the wall of the blood vessel. The stent sensor 200 components are individually clipped to the alternating stent segments 300. The individual components are interconnected to each other by a sufficient set of wires, bundled and coated by parylene or a similar barrier, which has been proven to be a long-term tissue tolerant material protecting against corrosion and serving as an electrical insulator.

The stent 100 is inserted into a narrowed blood vessel such as a coronary artery or other vessel requiring treatment for occlusion. The sensor 200 is capable of estimating the blood flow for early detection of narrowing vessel deposits that reduce the blood flow through the vessel. Moreover, this flow-control permits monitoring the need for interaction as well as controlling drug treatment for deposit reduction. The sensor 200 and stent 100 provide valuable information about blood flow through a coronary artery stent 100 in situ.

The sensor operation is as follows: the stent flow sensor 200 is built into a small segment of approximately 5-10 mm length of the stent 100 wall. Two ultrasound transducers 212 and 214 measure the blood flow through the stent 100. The transducers 212 and 214 are driven from integrated electronics mounted on flexible tape and molded into the stent 100 wall. Electric power is supplied via an inductive coil 206, inserted and molded as well into the stent 100 wall. Blood flow information is then transmitted via mechanical vibrations from the outer stent surface to the extracorporeal monitor unit 204.

The stent sensor 200 provides the following four advantages over known methods: 1) inductive energy transfer; 2) highly integrated electronics—provides power management and control; 3) ultrasonic stent flow measurement including transducer; and 4) transmitting of valuable information using mechanical wave oscillators—backed by a very basic model using a bi-morph bending principle.

Each of the above four benefits is related to a major stent sensor component. Each component of the stent sensor 200, in turn, can be independently utilized for other applications.

The operational principle is described using a particular stent design shown in FIG. 1. This design of stent meshing and dimensions is utilized as an example only. The stent flow sensor 200 as described can also be integrated into other designs with appropriate modifications, in accordance with the spirit and scope of the invention.

An embodiment of the present invention measures blood flow using an ultrasound Doppler arrangement attached to the mesh forming the tube wall of the stent 100, powered by the components (206 and 208), which are clipped to the stent 100. Powering the components 206 and 208 is obtained by a low frequency inductive voltage source (receiving coil 206) driven from an external magnetic field generator 218 (including a rectifier and capacitor and other components, not shown), regulated by the power processing unit 208, which also includes a driving oscillator providing the driving voltage for the Doppler transducer 212 as well as the clock for the signal processing unit 210. In addition, it provides power for all signal processing hardware and information transmitters 216 integrated into the stent 100.

The Doppler shifted backscatter is obtained by a Doppler receiver transducer 212 and 214. The frequency shift information is then extracted by the analog circuit 210, which in turn is transmitted via the transmitter 216 to the outside receiver 220.

Detecting critical flow conditions requires estimating the absolute flow-rate (ml-blood/sec.) through the stent cross-section. Because the Doppler primarily detects the blood flow velocities, a second parameter is required such as signal strength of the Doppler shifted signal. A separate module 210 detects the intensity of the Doppler shifted signal, which is also transmitted to the outside. The external information processing unit 222 processes the data, by calculating flow rates from flow profile application and mean velocity information. This is often done in standard spectral Doppler devices. The spectral content of the Doppler device is also utilized for estimating flow profiles.

In this embodiment, the stent sensor 200 estimates blood flow when the external magnetic field source is present, thus an electrical energy storage component (e.g., a battery) is not required. A capacitor may nevertheless provide a useful temporary energy source. However, it is also feasible to add such a suitable storage element, to allow flow measurement over a time period, where no magnetic field is present as the primary energy source. The use of the external source allows higher voltages driving the sensor 200 and shall provide enough energy driving acoustic transmitters for transferring the desired information to the extra corporeal unit 204. Reading the blood information can be achieved in two ways:

A.) the analog properties of frequency shift and signal intensity are encoded into the transmitter 212 in an analog manner. For example, the Doppler shift frequency and the Doppler signal amplitude directly modulates frequency and strength of a mechanical oscillator transmitting to the extra corporeal unit.

B.) Doppler signal data are processed within the stent unit 202 and then encoded into a signal to be transmitted, for example using Barker encoding and detected via cross-correlation by the outside unit 204.

While alternative A) provides a simpler implementation, alternative B) requires more hardware within the stent unit 202 but is much more reliable. Both solutions must be evaluated individually using an outer inductive coil of approximately 50 mm in diameter driven at a frequency range of 10-40 MHz which can be utilized to provide sufficient magnetic field strength up to a depth of 90 mm into the human body. The outer extra-corporeal part 204 of the sensor 200 includes an oscillator, a power supply, and the transmitting coil 218. Such a system can provide up to several mW at a regulated voltage of 3 V on a receive coil.

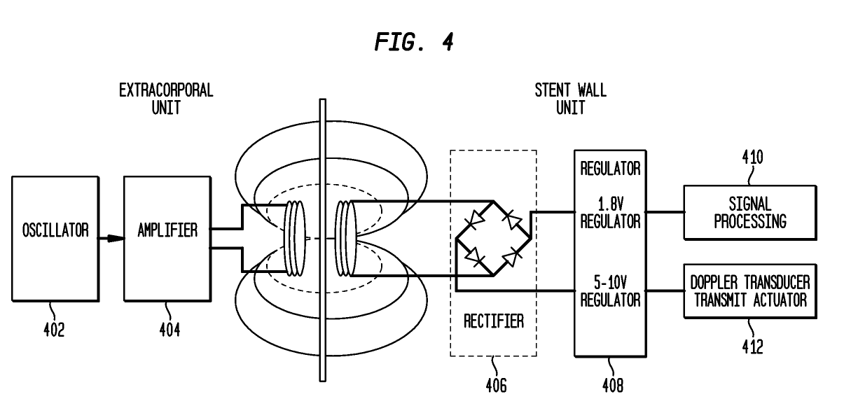

Referring to FIG. 4, there is shown a top level block diagram for a power supply for use with the invention. The extracorporeal unit 204 includes an oscillator 402 that produces a periodic oscillation signal which is amplified by an amplifier 404. The amplified signal is input into a transmitter coil that induces a current in the receiving coil 206 in the stent wall unit 202. The received current is rectified by a rectifier 406 and a voltage regulator 408 provides a 1.8 volt voltage for a signal processor 410 and a 5-10 volt voltage for a Doppler transducer transmit actuator 412.

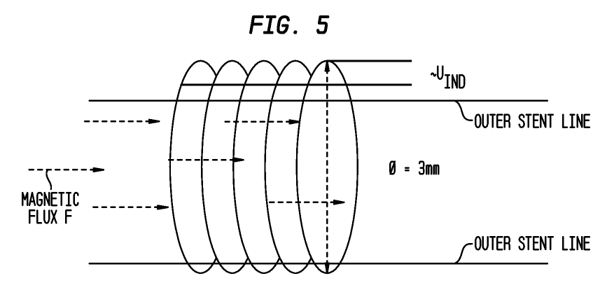

For the receive site, the intra corporeal pick up coil 206 is completely wound around the stent wall as shown in FIG. 5. To assure the coil turns are parallel to each other, the coil is prefabricated in a flexible parylene coated arrangement, intersecting the stent 100 at a flat angle prior to stent placement. When the catheter is inflated, thus pressing the stent 100 against the vessel wall, the coil moves into a more perpendicular position on the stent axis. In order to improve the efficiency of the coil, an electrical capacitor—residing in the power regulating compartment—is connected in series with the coil forming an electrical resonator circuit. If the driving magnetic field frequency corresponds to the resonance frequency of the circuit the energy transfer and in turn the power supply for the stent sensor 200 is maximized. The following calculation is an example of realization; other parameter sets can be utilized as well.

The voltage induced in the receiving coil is defined by:

U_{ind}=−NdΦ/dt=−N/1μᵣμ₀ωB₀dA sin(ωt) Eq. (1)

where N denotes the number of turns (N=10, in the example), Φ the magnetic flux, B₀ the magnetic flux density, ω the angular frequency of the signal, dA is the cross-sectional area of the receive coil, μ₀ is the magnetic permeability of free space, μᵣ the relative permeability of the material inside the coil, and 1 mm the coil length. To obtain an inductive voltage U_{ind} of approximately 100 [mV] a magnetic flux density of B₀˜2.5[mT] is required. For N=10 turns, an internal coil area of 3 mm diameter, a coil length of l=0.2 mm and a frequency of 20 MHz is assumed. This is just an example and not meant to be a value driving an actual sensor.

For this arrangement a suitable “energy tank capacitor” is derived by estimating the self inductance L of the pick up coil using the relation:

L=μμ₀N²dA/ Eq. (2)

using the parameter set given above L is obtained as L=5.56+10⁻⁷ henry

Using a tank capacitor of C₀=100 pF a resonance frequency of fᵣ=1/(2 π*sqrt (L C)) a resonance frequency fₛ=21 MHZ is achieved. It is noted that mounting a capacitor into the stent wall is easily achieved by using a high dielectric material (moldable ferroelectric coated by two electrodes).

While eq. (1) defines the induced voltage in the coil, with no current withdrawn (open loop), the actual current being utilized as power supply depends on the internal DC resistance of the coil, which is largely given by the coil material resistance and geometry. These factors include specific material conductivity, wire diameter and wire length. At high frequencies (MHz range) additional parameters such as:

a) operating frequency; b) skin effect; and c) mutual coupling between turns caused by eddy currents need to be considered.

These influences are difficult to predict analytically, hence experiments or FEM (finite element method) modeling has to be utilized to predict them correctly. However, measurements performed on arrangement of similar geometry and diameters reveal an Ohmic wire resistance to be expected of R=0.1 to 1 ohm.

Quality Factor Q of the Resonance Coil.

The gain in current to be expected from using the resonator arrangement depicted below is quantified by the quality factor Q of the resonance circuit of the pick-up coil. Q is calculated from:

Q=2*π*fₛ*L/Rₛ, with fₛ=21 MHz, L=5.56*10−7 and R=0.1-1 [Ohms] a Q ranging from Q=70-700 is expected.

Power Recovery Module.

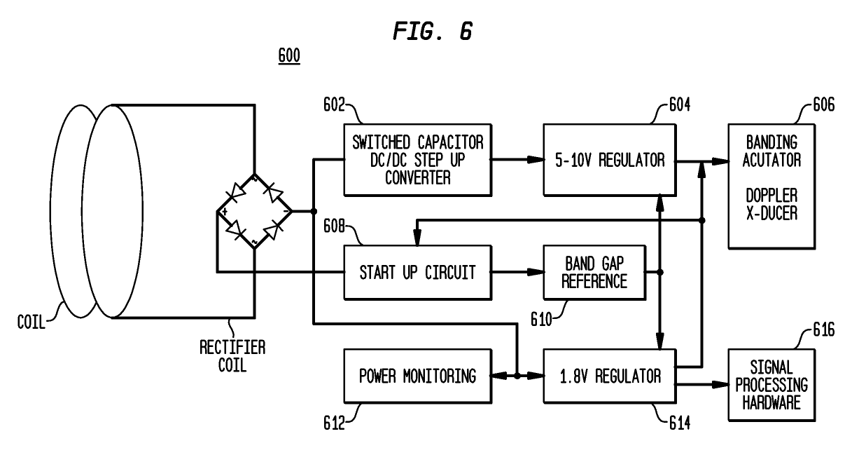

Referring to FIG. 6, the voltage from the receiving coil 206 is rectified and regulated for driving the Doppler unit and the transmitting actuator as well and will supply the signal processing electronics.

The power recovery unit basically includes a full wave rectifier unit 601 and two regulator units (604 and 614) providing two different regulated voltages of 1.8 V for the signal processing ASIC (application specific integrated circuit) and possibly higher voltage 5-10 V for the Doppler device 412 and the transmitting actuator.

The functional components within this unit are:

start up circuit 608—waking up all other components to be active;

bandgap reference 610 setting the limits for maintaining constant voltage and the 1.8 V;

voltage regulator 614;

power monitor block 612;

step up DC-DC converter 602; and

5-10 V regulator 604.

Clock (not shown).

The clock providing the timing for all circuits including driving the ultrasound transmitter is another main component of the internal stent circuitry. This is a non-trivial part as state of the art clock oscillators are by far too large to be placed within the stent wall. Therefore, a new approach is considered by building the clock as a MEMS component. The clock design will follow directions as outlined for cell phone components.

Stent Flow Sensor Basic Considerations.

Blood Flow Velocities.

As a rough estimate of flow and velocity ranges within the coronary artery the following assumptions are made:

typical cardiac output (CO) in an adult: 5-30 l/min.

Coronary supply 4% of CO: approx. 0.2-1.2 l/min.

Average in each of five major coronary arteries: CA flow 40-240 ml/min or 0.7-4 ml/sec.

Internal diameter: approx. 2 mm average (1-3 mm variation).

Average cross-section: A˜0.03 cm2

Estimated blood velocity: v=CAflow/A˜20-100 micron/s or μm/s

Since the stent flow sensor 200 as shown is a MEMS (Micro Electro Mechanical System) device all dimensions will be stated in μm.

Stent flow will be detected and quantified in terms of peak velocity and temporal average using the Doppler principle. Although pulse-echo Doppler could be utilized, the focus here is on CW (continuous wave) Doppler with separate transmitter and receiver adjacent to each other or co-focal. The stent curvature provides a natural overlapping area for both the transmitted wave field as well as the receiving sensitivity field. The Doppler frequency shift obtained from ultrasound backscatter from the RBCs (red blood cells) is obtained from the well-known Doppler equation.

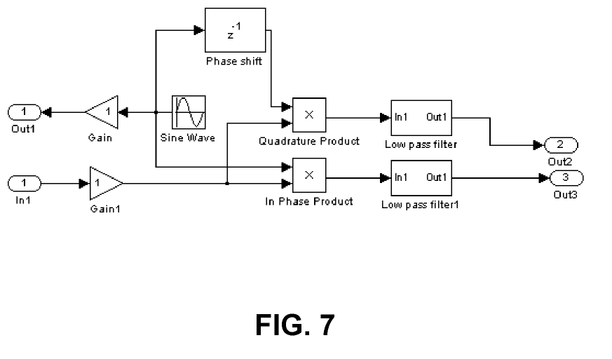

FIG. 7 depicts the basic block diagram for the CW Doppler system implemented in a high density IC package. Together with the transducers described below this is the central part of the system. In addition to the fact that a CW-Doppler system is a very stable system with the fewest components compared to other Doppler arrangements, e.g. Pulse Doppler, the fact that the analog signal output is a low frequency (up to 30 kHz) appears very useful. Since this frequency can be directly used for driving the mechanical outward vibrator no additional down-sampling or up-sampling would be required. The Doppler output is directly fed into an amplifier driving the outward oscillator. In this case one Doppler output channel would be dropped and only the forward flow would be registered and transmitted to the outside.

The decision about aperture and frequency requires balancing between the following factors:

frequency dependent ultrasound attenuation in blood;

frequency dependent ultrasound scatter cross-section of red blood cells (RBC);

received Doppler signal strength;

maximizing field overlap (transmit beam/receive beam);

angle between blood flow vector and transmit beam axis; and

insertion loss due to various transmitter/receiver properties and geometry.

Proposed Transducer Arrangement.

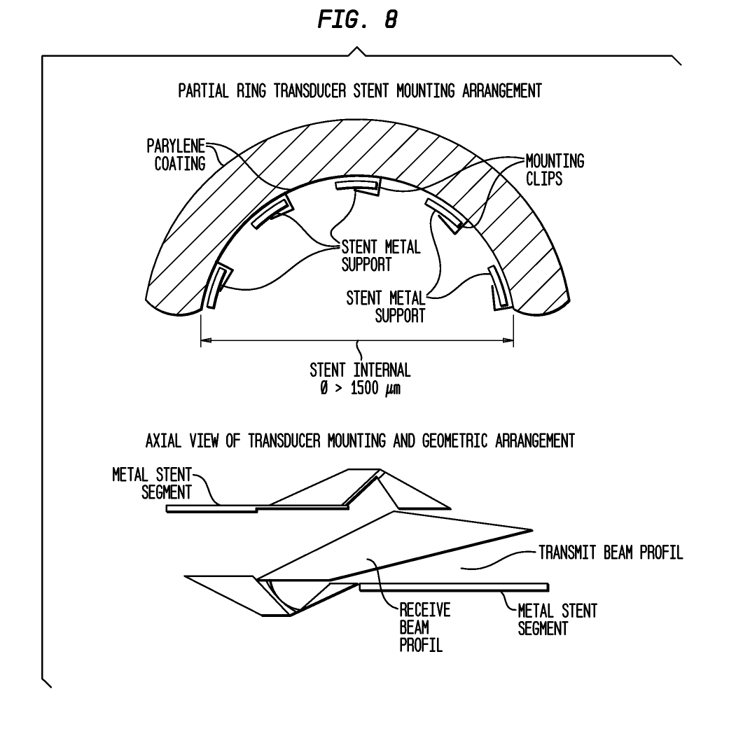

The transmit/receive transducer unit consists of two half annular segments embedded in a polymer material while sustaining an angle of approximately 45 degrees to the stent longitudinal axis 106 as shown in FIG. 8. The shape of the polymer transducer housing provides a flat profile for smoothly fitting to the inner vessel wall. In addition, the polymer housing may serve as a reservoir for releasing drugs, thus minimizing tissue proliferation.

Since the angle between beam axes and flow direction or stent axis shall be optimized, an oblique angle between the stent axis and the aperture elevation is required. If the enlargement beyond the stent diameter is limited to 500 μm, and an elevation angle of approximately 45 degrees is utilized, which yields about 70 percent of the maximum obtainable signal strength, the maximum elevation of the aperture del (δ)=500*sqrt(2)˜700 μm. Assuming the application requires approx. 10λ (wavelengths) for reasonable focusing, the resulting frequency is approximately 20 MHz.

Assuming the two half annular sections are to be arranged for transmit and receive around the stent 100 as depicted in FIG. 8, yields up to 2350 μm circumferential length should be used as an azimuthal aperture for both the transmitter and the receiver. From these design definitions the maximum transducer area would be

A_{Tx}, A_{Rv}˜700 μm×2350 μm

at a center frequency of 20 MHz (10-30 MHz). The azimuth length can be reduced to about 750 μm providing a more symmetric beam profile, which might still be sufficient for measuring blood flow at a very close range. However, with the presented design an aperture up to three times larger is available if necessary for signal to noise improvements.

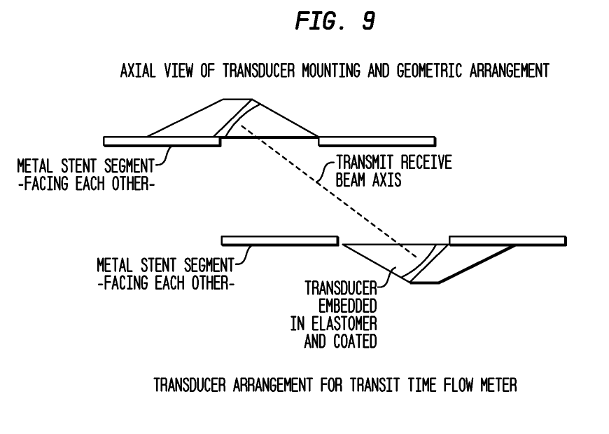

An alternative approach to the Doppler transducer arrangement is presented in FIG. 9, wherein a transit time flow meter is presented in an arial view of the transducer mounting and geometric arrangement. Although the polymer housing of the transducer provides enough protection to the rather brittle and therefore sensitive piezoelectric (pzt) element, the transducer can be constructed from 1-3 piezo composites. With pzt pillars, 40 μm in diameter, embedded in an elastomer matrix of approximately 50 μm pitch. The elastomer matrix provides a flexible structure by material properties.

The following transducer specifications are based on typical PZT properties e.g. PZT 5HA or PZT 5HB for rough calculations. A separate calculation for 1-3 composite is not yet given, but it is easily derived from full pzt properties by considering a fill factor of 60%, which is the percentage of ceramic PZT within the composite.

Transducer Thickness:

Assuming a thickness frequency constant of 1500 (mm kHz) the expected thickness for 20 MHz is 75 μm and 150 μm and 50 μm for 10 MHz and 30 MHz center frequency, respectively.

Transducer Impedance at Serial Resonance.

Transducer impedance is calculated using the capacitance impedance at center frequency and geometric and permittivity properties of PZT (lead zirconate titanate). According to

R_{fc}=abs(1/(2*π*f_{c}*C₀)) with C₀=∈_{rpzt}*∈₀*A/t_{Tx}

with t_{Tx} the transducer thickness. Where fc is the center frequency of the transducer, C₀ the capacitance of the transducer, ∈_{rpzt} the relative dielectric constant of the transducers PZT material, A is the transmitting aperture area of the transducer and t_{Tx} the transducer thickness. Using ∈_{rpzt}=1500 and the thickness from above for 20 MHz (10 MHz, 30 MHz) yields:

C0=6.2 pF, 12.4 pF and 18.6 pF for 10, 20 and 30 MHZ respectively, resulting in R_{fc}=1285 Ohm, 321 Ohm, and 143 Ohm, respectively.

Equivalent Electrical Circuit.

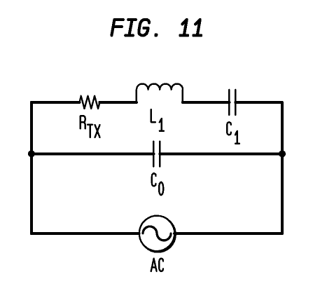

Referring to FIG. 11, a transmit/receive transducer equivalent circuit is shown. Since the above values represent the serial impedance for a matched transducer, the values for unmatched transducers are obtained by dividing the above values by Q*k², where Q is the mechanical damping and k² is the effective electro-mechanical coupling coefficient.

Considering a Q of 10-50, higher Q is preferable for resonating at serial resonance for CW Doppler application, and k²=0.25 leads to a ratio of 2.5 to 12.5 to be considered. Therefore, the final range for transducer impedance ranging from 10-30 MHz and Qm from 10-50 is R=[10-500] Ohm.

This range is well-handled by standard ultrasound electronics. Using Rtx and C0 as well as a reasonable value for k2eff=0.25 the equivalent circuit parameters are obtained. For example, at 20 MHz:

C0=12.4 pF

C1=3.72 pF

L1=17 mHy

R1=20-100 Ohms

Assuming a thickness of 75 μm for the transmitter (f0=20 MHz) and voltage amplitude of 10 [V] the resulting pressure amplitude is approx. 4 [bar]. Due to the loss at the transducer/blood interface the resulting pressure is ˜120 [mbar]; using a single matching layer the achievable pressure amplitude is ˜1 [bar].

Bulk signal amplitude loss due to attenuation taking into account absorption and scatter from blood: for a quick estimate a signal drop of 50 dB is assumed.

Vₜ=k_{eff}²*Vt˜0.25 V for 1 V transmit voltage. Considering the 50 dB scattering drop yields: Vr˜500 uV. A little different approach using the d33 piezo constant yields ˜750 μm.

While the above parameters are based on solid PZT, corrections have to be made using the equivalent properties for 1-3 composites based on Smith and Auld calculations (see: M. R. Shah et al.: A study of printed spiral coils for neuroprosthetic transcranial telemetry applications IEEE BME Trans, Vol. 45, No. 7 1998, p. 867-876).

Inward Power Transmission.

The issue of power supply is best addressed by using power MEMS design. Micro Seismic Power Generator Using Electret Polymer Film describes the basic principle and design calculations. This type of power generator relies on the mechanical energy driving the electret membrane and converts therefore mechanical energy into electrical. Inductive power generation is most likely the better way, as it appears more effective in terms of electrical power generated.

Inductive coil to coil power transmission is the preferred procedure for transmitting energy from the outside to the implanted stent sensor. The use of electret microphones and the piezoelectric energy conversion turn out to be an alternative to the advantage of coil to coil is at least twofold:

1. The energy transfer is more easily adjusted to the power needs.

2. There is no mechanical energy transfer involved, which could interfere with the read-out transmission which is considered using mechanical vibration and field transmission as a means of communication.

A Transcutaneous Energy Transfer (TET) system consisting of the outer coil and stent integrated receiving coil can be simulated under real world conditions using Finite Element Modeling (FEM). Since larger distances between the outer and inner coil have to be considered, the model requires either separating the two coil models, by using the output of the outer coil as an input for the integrated coil. Or, by using Border Element Modeling (BEM) where the outer coil is modeled extending up to a well-defined surface and then the field is extended as usual by Green functions into the inner coil. This second approach allows for inclusion of magnetic tissue parameter variations, if any.

### Outward Information Transmission

While power transfer from the outside into the stent 100 is performed via a magnetic field, the magnetic field approach does not appear useful for transmitting blood flow information from the stent to the outside system. In addition, if applied, both fields may interfere, as they would be generated synchronously. A mechanically vibrating system is used for the outward transmission task. The approach hereto is twofold: in one embodiment the vibrator creates sufficient acoustic wave energy for a wave traveling from the stent to body surface and will be detected by a suitable receiver. In a second embodiment the external receiver is focused onto the stent and detects using gated pulse echo information the BF (blood-flow) information from the sensor. In this case a high acoustic reflectivity surface will vibrate and therefore supporting the task.

In case the blood flow information is encoded into a transmit signal, the resonance frequency and carrier bandwidth are selected according to stent geometry and useful material (preferred solutions). In case an analog signal processing scheme is utilized the vibrator is required to vibrate in the lower ten kHz range, which is the range of the Doppler shift.

When the stent flow ultrasound sensor estimates the blood flow through the stent 100, the information has to be communicated to the world outside the patient. As a first choice it is proposed to use the magnetic coil principle for this task. The procedure is similar to power transmission and follows the same steps outlined in that context. As an alternative to the magnetic coil transmission may be accomplished via mechanical vibrations. In this case a section of the stent 100 will vibrate with encoded flow information. This vibration can either be picked up by a sensor placed in the line of sight—acoustically—on the patient's skin or the stent sensor transmitter is focused onto the outside receiver. Also both ways can be combined into an optimal transmit system. Two ways of generating the vibration transmitter are used as exemplary embodiments:

a) vibrating cylinder mode; and

vibrating half cylinder mode (or half pipe mode). There are two major constraints to consider: the size of the vibrator and the energy available for driving the transmitter.

The size ultimately defines the frequency and power available the strength of the signal transmitted. For two reasons the frequency should be chosen as low as possible: attenuation and center frequency; and bandwidth required to encode the information content being transmitted. The second one appears to be resolved easily, as the information potentially is encoded into 1 bit train of transmit and no-transmit.

Due to the size constraints, directly transmitting from a piezo element requires high frequencies, in the order of one MHz and higher, with high attenuation associated with it. Therefore a low frequency resonator is considered more suitable for transmitting.

Operational Principle.

The basic idea for a low frequency system results from the potential of bending resonators being used, which provide large displacement when compared to single element piezo-transmitter. The low frequency density waves can travel to the body surface for being detected by the receiver sensor. However, also the receiving sensor could be an ultrasound array system (e.g. electronic phased array) which may focus directly on the vibrator, where it measures the transmitter displacement via cross-correlation of subsequent fired signals.

However, estimating the vibration via cross-correlation requires displacement amplitude substantially larger as 1.5 μm, the resolution which can be detected via time shifts in subsequent signals. This constraint results from a time of flight resolution of 1 ns, a typical value for biomedical ultrasound. For sufficient SNR (signal to noise ratio) approximately 10 μm in displacement amplitudes will be required.

Even at much lower displacement amplitudes, the associated pressure is quite high due to the incompressibility of tissue or body fluid respectively. Although this pressure is in the range of biomedical ultrasonic imaging, the spread from the source to the distant receiver has to be accounted for. At 10 kHz the wavelength is 15 cm thus the transmitter is de facto a point source with quadratic amplitude loss on distance. This loss is partially recovered by a long transmitting antenna, which is implemented by using the length of stent as the vibrating antenna.

Bimorph Bender

A slab of PZT, 0.500 μm wide, 100 μm thick and 500 μm long is attached to a steel slab of the same size. A voltage is applied to the bender at the piezo slab electrodes in the direction of the poled piezo-axis which is the thickness axis. A driving voltage compresses or stretches the piezo-element associated with a corresponding stress/compression in the axis along the slab. Using a first numerical model the bimorph slab is kept in a fixed position, while another slab remains freely moving.

The experiment was carried out with the following parameters:
Length: 500 μm
Width: 500 μm
Thickness:
   PZT: 100 μm
   Steel: 100 μm

At field strength of 2 KV/mm, equivalent to 1000 V/mm the maximum bending is 150 μm. A bending actuator of similar geometry will very likely provide enough displacement amplitude for ultrasonic pick up. Even the arrangement implemented will be different with both sides remaining fixed in the lateral stent axis and only sliding with respect to the long stent axis. To circumvent a high voltage it is proposed to build the piezo-part of the bender from N-layer electrically connected in parallel, which allows cutting the drive voltage to 1/N but remaining the full electrical field strength of 2 KV/mm.

It is noted the range of dimension under the constraints of the stent dimension permits the bender driving frequency associated with the Doppler shift frequency. The bender could be driven with the Doppler shift frequency as the oscillator. However, the bandwidth shown here is relatively small and therefore not wide enough to cover the frequency range of the Doppler shift enough for the blood flow diagnosis.

Therefore, while there has been described what is presently considered to be the preferred embodiment, it will be understood by those skilled in the art that other modifications can be made within the spirit of the invention. The embodiments and graphical illustrations, as described, were chosen in order to explain the principles of the invention, show its practical application, and enable those with ordinary skill in the art to understand how to make and use the invention. It should be understood that the invention is not limited to the embodiments described above, but rather should be interpreted within the full meaning and scope of the invention.

1. A stent for placement in a blood vessel of a patient, the stent comprising:
a proximal end;
a distal end;
a generally circular cross-section with a diameter;
a plurality of interlocked stent segments to form the stent;
a Micro Electro-Mechanical System (MEMS) ultrasound sensor using a Doppler principle for determining patency and flow rate of blood through the cross-section, and
pressure drop from the proximal end to the distal end;
a transmitter for providing signals to an ex-situ receiver; and
a coil device coupled with the transmitter for powering said transmitter with energy received from outside a body of the patient.

2. The stent of claim 1 further comprising a receiver sensor placed on the patient's skin wherein vibration from the transmitter is picked up acoustically by the receiver sensor.

3. The stent of claim 1 wherein the transmitter comprises a vibrator providing mechanical vibrations for transmitting information to a location outside the body of the patient.

4. The stent of claim 1 further comprising a receiving sensor located outside the body of the patient to receive information from the transmitter.

5. The stent of claim 1 wherein the transmitter comprises a magnetic coil producing vibrations to communicate information determined by the sensor to a unit outside the patient.

6. The stent of claim 1 further comprising an optimal transmission system comprising a combination of a vibrating cylinder mode and a vibrating half cylinder mode.

7. The stent of claim 1 wherein the transmitter comprises a piezo element.

8. The stent of claim 1 wherein the transmitter transmits at low frequency, not exceeding 3,000 kilohertz.

9. The stent of claim 8 wherein a plurality of bending resonators are used in the transmitter.

10. The stent of claim 7 wherein the transmitter transmits from the piezo element at frequencies of one megahertz and higher.

11. The stent of claim 10 wherein estimating a vibration is done by cross-correlation.

12. The stent of claim 1 wherein the stent is for placement in an artery.

13. A method of detecting fluid flow through a stent embedded in a patient's blood vessel, said method comprising:
placing the stent in the patient's blood vessel, said stent comprising:
a proximal end;
a distal end;
a generally circular cross-section with a diameter;
a plurality of interlocking stent segments to form the stent;
a Micro Electro-Mechanical System (MEMS) ultrasound sensor using a Doppler principle for determining patency and flow rate of blood through the cross-section, and pressure drop from the proximal end to the distal end;
a transmitter for providing signals to an ex-situ receiver; and
a coil device coupled with the transmitter for powering said transmitter with energy received from outside a body of the patient;
inflating the stent such that the interlocking stent segments lock together;
using a Micro Electro-Mechanical System (MEMS) ultrasound sensor for determining patency and flow rate through the stent, and pressure drop from the proximal end to the distal end of the stent;
using the transmitter in the stent for providing signals to an external receiver outside the patient's body; and
using the coil device for receiving energy from outside the patient's body and for powering the transmitter.

14. The method of claim 13 further comprising placing a receiver sensor on the patient's skin and acoustically picking up vibration from the transmitter.

15. The method of claim 13 further comprising placing a receiving sensor outside the body of the patient to receive information from the transmitter of the stent.

16. The method of claim 13 further comprising producing vibrations with the transmitter having a magnetic coil to communicate information determined by the sensor to outside the patient.

17. The method of claim 13 wherein the transmitter comprises a piezo element.

18. The method of claim 13 wherein the transmitter transmits at low frequency, not exceeding 3,000 kilohertz.

19. The method of claim 13 wherein a plurality of bending resonators are used in the transmitter.

20. The method of claim 13 wherein the transmitter transmits from piezo elements at frequencies of one megahertz and higher.
